(19) United States
(12) Patent Application Publication
(10) Pub. No.: US 2024/0299338 A1
(43) Pub. Date: Sep. 12, 2024

(54) COMPOSITIONS FOR TREATMENT OF VAGINAL ATROPHY

(71) Applicant: Innocan Pharma Ltd., Herzliya (IL)

(72) Inventor: Nir AVRAM, Metar (IL)

(73) Assignee: Innocan Pharma Ltd., Herzliya (IL)

(21) Appl. No.: 18/569,233

(22) PCT Filed: Jun. 12, 2022
(86) PCT No.: PCT/IL2022/050625

§ 371 Date: Dec. 12, 2023

Related U.S. Application Data
(60) Provisional application No. 63/210,024, filed on Jun. 13, 2021.

(51) Int. Cl. A61K 31/352; A61K 9/00; A61K 31/00; A61K 31/716; A61K 47/36; A61P 15/02

(52) U.S. Cl. CPC A61K 31/352; A61K 9/0034; A61K 31/658; A61K 31/716; A61K 47/36; A61P 15/02

(57) ABSTRACT

Described herein are compositions which can be used to alleviate vaginal dryness and vaginal atrophy. The compositions comprise a cannabinoid and a phytoestrogen. Optionally, the composition further comprises a prebiotic agent, preferably α-glucan oligosaccharide. Optionally the composition further comprises a lubricating agent, preferably hyaluronic acid.

### CROSS REFERENCE TO RELATED APPLICATIONS

Benefit is claimed to U.S. Provisional Patent Application No. 63/210,024 filed Jun. 13, 2021; the contents of which are incorporated by reference herein in its entirety.

### FIELD

Provided herein are compositions comprising cannabinoids for treatment of conditions including vaginal dryness and vaginal atrophy.

### BACKGROUND

Vaginal atrophy is a condition associated with loss of moisture, thinning, and inflammation of the vaginal walls, which can be associated with decrease in estrogen levels in women, often associated with menopause. It can lead to itching and painful intercourse. Although menopause can decrease estrogen levels in women thereby causing vaginal dryness, other factors may cause vaginal dryness such as certain medications such as antihistamines, antidepressants, and chemotherapeutic treatment.

### SUMMARY

Described herein are compositions which can be used to alleviate vaginal dryness and vaginal atrophy. The compositions comprise a cannabinoid and a phytoestrogen. Optionally, the composition further comprises a prebiotic agent, preferably α-glucan oligosaccharide. Optionally the composition further comprises a lubricating agent, preferably hyaluronic acid.

The foregoing and other objects, features, and advantages will become more apparent from the following detailed description.

### DETAILED DESCRIPTION

Unless otherwise noted, technical terms are used according to conventional usage. Definitions of common terms in pharmaceutical sciences can be found in Troy et al. Remington: The Science and Practice of Pharmacy. Published by Lippincott Williams & Wilkins, 2006. In case of conflict, the present specification, including explanations of terms, will control. In addition, all the materials, methods, and examples are illustrative and not intended to be limiting.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

### Definitions

Cannabinoid: A cannabinoid is a chemical compound that acts on cannabinoid receptors in cells in mammals, including in humans. Cannabinoids can be manufactured synthetically or obtained from various parts of the genus Cannabis, in particular, from the species Cannabis Sativa. Cannabinoids from the cannabis plant are referred to as phytocannabinoids. Two preferred cannabinoids according to various embodiments, are (−)-trans-Δ⁹-tetrahydrocannabinol, and/or isomers thereof (THC) and cannabidiol (CBD). Alternatively, a cannabinoid may be in the form of cannabis extract. Alternatively, a cannabinoid may be in the form of a synthetic cannabinoid. Compositions described herein may comprise one cannabinoid or multiple cannabinoids, such as a combination of CBD and THC.
Phytoestrogen: A compound, from a plant source, which acts on the estrogen receptor in humans. The phytoestrogen may be an isoflavone.
Prebiotic: A compound that promotes the growth of beneficial microorganisms on or in the body of a host. Certain prebiotics in foods include oligosaccharides which are beneficial to host gut bacteria when consumed by the host.

### DESCRIPTION OF EMBODIMENTS

Described herein, according to some embodiments, are compositions comprising a cannabinoid and a phytoestrogen. Some embodiments refer to compositions comprising a cannabinoid and a prebiotic agent preferably an α-glucan oligosaccharide. Some embodiments refer to compositions comprising a cannabinoid and lubricant agent preferably hyaluronic acid.

Compositions described herein may be formulated as an emulsion, a moisturizing gel, a cream, an oil, a lotion, a liquid, or a lubricating gel.

Compositions described herein may further comprise a plant-based oil. Optionally, the plant-based oil is selected from the group consisting of: hempseed oil, almond oil, sesame oil, shea butter, coconut oil, and hippophae oil.

The amount of cannabinoid may range from between 0.1% by weight to 5% by weight, preferably between 1% to 2% by weight.

The amount of phytoestrogen may range from between 0.003% by weight to 0.015% by weight.

The amount of prebiotic α-glucan oligosaccharide may range from between 0.5% by weight to 5% by weight, preferably 1% weight.

The amount of hyaluronic acid may range from between 0.01% by weight to 1% by weight, preferably between 0.3% to 0.5% by weight.

A single cannabinoid or a mixture of cannabinoids, such as a cannabis extract, comprising multiple cannabinoids may be used in compositions described herein. Optionally, the cannabinoid used is cannabidiol.

Optionally, the compositions described herein further comprise glycerin, preferably in an amount between 5% and 40% by weight. Optionally, the compositions comprise water, preferably in an amount of 50% or more water by weight.

Administration of the compositions described herein may be performed on a daily basis, optionally, twice, three of four times daily, in patients in need thereof. Optionally, the compositions may be applied to the vaginal area before intercourse, to add moisture and prevent pain.

The amount administered per application for a moisturizing composition may vary from between 1-5 grams daily.

Additional ingredients in compositions described herein may include one or more than one of the following: Panax ginseng root extract, allantoin, squalene, Olea europaea (olive) fruit oil, Persea gratissima (avocado) fruit oil, hippophae rhamnoides (sea buckthorn) fruit oil, apricot (Prunus armeniaca) kernel oil, tea tree (Melaleuca alternifolia) leaf oil, aloe vera (aloe barbadensis) leaf extract 200:1 powder, Cocos nucifera (Coconut) Oil, Lactic acid, Simmondsia chinensis (Jojoba) Seed Oil, Tocopheryl Acetate, Tocopherol, Panthenol, Niacinamide, Helianthus annuus (Sunflower) Seed Oil or Carthamus Tinctorius (Safflower) Seed Oil.

Compositions described herein are designed to have an immediate effect, to provide moisture and lubrication when administered and alleviate pain and discomfort, and a long-term effect, of providing moisture even many hours after administration. It is suggested that the long-term effect may be contributed to by the phyto-estrogen, which can have a moisturizing effect continuing for 24 hours after administration.

It is suggested that compositions described herein are beneficial in treating vaginal atrophy. Cannabinoids, in particular CBD, may have a synergistic effect in the treatment of vaginal atrophy when used in combination with an additional agent as described herein.

The following examples are provided to illustrate certain particular features and/or embodiments. These examples should not be construed to limit the disclosure to the particular features or embodiments described.

### EXAMPLES

### Example 1

A lubricating gel comprising CBD is prepared using the ingredients in table 1:

TABLE 1

| | | Amount |
|---|---|---|
| | | (percent by |
| Ingredient | Function | weight) |
| Water | Filler | 58.92 |
| Glycerin | Humectant & Moisturizer | 30.00 |
| Hyaluronic acid | Lubricating Agent | 0.50 |
| Butylene glycol | Humectant & Moisturizer | 5.00 |
| Sodium Benzoate | Preservative | 0.25 |
| Hydroxyethylcellulose | Gelling Agent, Thickener | 0.80 |
| Lecithin (and) Soy | Phytoestrogen | 2.00 |
| Isoflavones (and) | | |
| Polysorbate 80 (and) | | |
| Alcohol (and) Aqua | | |
| α-Glucan oligosaccharide | Prebiotic | 1.00 |
| Cannabidiol (plant isolate | Pain relief, anti- | 1.50 |
| having greater than 97% | inflammation agent | |
| cannabidiol) | | |
| Glycolic Acid | pH adjustment agent | 0.03 |

The first four ingredients are added to a main vessel and mixed well until a homogenous gel is obtained. Sodium benzoate is then added, then hydroxyethylcellulose, while heating to 70° C. while homogenizing. A clear gel without lumps is obtained, and homogenizing stops. Mixture is stirred slowly while cooling. When the mixture reaches 40° C., the following three ingredients are sequentially added, while mixing. Glycolic acid is added slowly until pH is tested and reaches between 4.5 and 5.0.

Lipobelle™ is a liposomal mixture of Lecithin (and) Soy Isoflavones (and) Polysorbate 80 (and) Alcohol (and) water, and is available from Mibelle, Switzerland. It contains the soy isoflavone genistein, which binds to the estrogen receptor in humans in an amount of 0.3% by weight. It is suggested that topical administration of a soy isoflavone, for example, genistein, provides a plant estrogen substitute giving benefits such as skin moisture, flexibility and elasticity, without the potential dangers associated with administration of estrogen.

Natrosol™ 250 HHX Hydroxyethylcellulose (HEC) by Ashland, PC Grade is a nonionic water-soluble polymer with a viscosity range of 3,400-5,000 cps. (1% aqueous solution)

α-Glucan oligosaccharide is marketed under the trade name Bioecolia® and produced by Solabia. It has been suggested that topical application of α-Glucan oligosaccharide protects and stimulates beneficial microbial skin flora without allowing for growth of pathogens and undesirable flora.

Without being bound by theory, it is suggested that hyaluronic acid provides long-acting moisture when applied to the vaginal area.

The lubricating gel prepared in this example may be used, for example, by applying before intercourse.

### Example 2

A cream comprising CBD is prepared using the ingredients in table 2:

TABLE 2

| | | Amount |
|---|---|---|
| | | (percent by |
| Ingredient | Function | weight) |
| Water | Filler | 65.40 |
| Glycerin | Humectant & Moisturizer | 10.00 |
| Hyaluronic acid | Lubricating Agent | 0.30 |
| Butylene glycol | Humectant & Moisturizer | 2.00 |
| Coconut oil | Emollient, Lubricant | 2.00 |
| C14-22 Alcohols (and) | Emulsifier | 2.00 |
| C12-20 Alkyl Glucoside | | |
| Shea Butter | Emollient, anti | 3.00 |
| | inflammatory and skin | |
| | healing | |
| Almond oil | Emollient, Lubricant | 3.00 |
| Bisabolol | Soothing and calming agent | 0.10 |
| Sesame oil | Emollient, Lubricant | 5.00 |
| Tocopheryl acetate | Antioxidant | 0.20 |
| Hempseed oil | CBD Solvent and emollient | 0.50 |
| Phenoxyethanol (and) | Preservative | 1.00 |
| Ethylhexylglycerin | | |
| Cannabidiol (plant isolate | Pain relief, anti- | 1.50 |
| having greater than 97% | inflammation agent | |
| cannabidiol) | | |
| Polyacrylate | Emulsifier & Thickener | 1.00 |
| Crosspolymer-6 | | |
| Lecithin (and) Soy | Phytoestrogen | 2.00 |
| Isoflavones (and) | | |
| Polysorbate 80 (and) | | |
| Alcohol (and) Aqua | | |
| α-Glucan oligosaccharide | Prebiotic | 1.00 |

Polyacrylate Crosspolymer-6 is obtained as Sepimax Zen™ which can be obtained from Seppic.

C14-22 Alcohols (and) C12-20 Alkyl Glucoside is obtained as Montanov™ L, which can be obtained from Seppic.

In a main vessel, the first 4 ingredients are added and heated while being homogenized to 75° C. In a separate vessel, the following 10 ingredients of the oily phase were added while heating to 75° C. until all solids and waxes are completely dissolved. The polyacrylate crosspolymer-6 is then added to the oily phase while mixing. The oily phase is then added to the main vessel at 75° C. and then homogenized for 10 minutes, then cooled while mixing at medium speed. When cooled to 40° C. the last two ingredients are sequentially added and the mixture is homogenized for another two minutes. Mixing and cooling is continued to 35° C. A white cream/lotion is obtained.

The cream produced by this example may be used for treating vaginal atrophy.

### Example 3

A moisturizing cream comprising CBD is prepared using the ingredients in table 3:

TABLE 3

| | | Amount |
|---|---|---|
| | | (percent by |
| Ingredient | Function | weight) |
| Water | Filler | 64.40 |
| Glycerin | Humectant & Moisturizer | 10.00 |
| Hyaluronic acid | Lubricating Agent | 0.30 |
| Butylene glycol | Humectant & Moisturizer | 2.00 |
| Coconut oil | Emollient, Lubricant | 2.00 |
| C14-22 Alcohols (and) | Emulsifier | 2.00 |
| C12-20 Alkyl Glucoside | | |
| Shea Butter | Emollient, anti | 3.00 |
| | inflammatory and skin | |
| | healing | |
| Hippophae oil | Moisturizer | 3.00 |
| Bisabolol | Soothing and calming agent | 0.10 |
| Sesame oil | Emollient, Lubricant | 5.00 |
| Tocopheryl acetate | Antioxidant | 0.20 |
| Hempseed oil | CBD Solvent and emollient | 0.50 |
| Phenoxyethanol (and) | Preservative | 1.00 |
| Ethylhexylglycerin | | |
| Cannabidiol (plant isolate | Pain relief, anti- | 1.50 |
| having greater than 97% | inflammation agent | |
| cannabidiol) | | |
| Polyacrylate | Emulsifier & Thickener | 1.00 |
| Crosspolymer-6 | | |
| Lecithin (and) Soy | Phytoestrogen | 2.00 |
| Isoflavones (and) | | |
| Polysorbate 80 (and) | | |
| Alcohol (and) Aqua | | |
| α-Glucan oligosaccharide | Prebiotic | 1.00 |
| Aloe vera gel | Moisturizer | 1.00 |

In a main vessel, the first 4 ingredients are added and heated while being homogenized to 75° C. In a separate vessel, the following 10 ingredients of the oily phase were added while heating to 75° C. until all solids and waxes are completely dissolved. The polyacrylate crosspolymer-6 is then added to the oily phase while mixing. The oily phase is then added to the main vessel at 75° C. and then homogenized for 10 minutes, then cooled while mixing at medium speed. When cooled to 40° C. the last three ingredients are sequentially added and the mixture is homogenized for another two minutes. Mixing and cooling is continued to 35° C. A white cream/lotion is obtained.

### Example 4

A moisturizing gel comprising CBD is prepared using the ingredients in table 4:

TABLE 4

| | | Amount |
|---|---|---|
| | | (percent by |
| Ingredient | Function | weight) |
| Water | Filler | 59.05 |
| Glycerin | Humectant & Moisturizer | 30.00 |
| Hyaluronic acid | Lubricating Agent | 0.50 |
| Pentylene glycol | Humectant & Moisturizer | 5.00 |
| Acrylates/C10-30 Alkyl | | 0.50 |
| Acrylate Crosspolymer | | |
| Lecithin (and) Soy | Phytoestrogen | 2.00 |
| Isoflavones (and) | | |
| Polysorbate 80 (and) | | |
| Alcohol (and) Aqua | | |
| α-Glucan oligosaccharide | Prebiotic | 1.00 |
| Cannabidiol (plant isolate | Pain relief, anti- | 1.50 |
| having greater than 97% | inflammation agent | |
| cannabidiol) | | |
| Triethanolamine | pH adjustment | 0.45 |

In a main vessel, the first 5 ingredients are combined and heated while being homogenized to 70° C. until a homogeneous gel without solids or particles is obtained. Homogenization is stopped and the mixture is cooled while mixing. At 40° C., the next three ingredients are sequentially added while mixing. Slowly triethanolamine is added until the pH reaches 6.0 to 6.5. A slightly translucent gel is obtained.

The aqueous moisturizing gel described in this example may be quickly absorbed in the vagina after application. It is intended for application to the vaginal area, either directly, or using an applicator configured to be inserted into the vagina.

Advantages of the compositions described herein include renewing moisture to the vaginal area while treating and preventing inflammation. Without being bound by theory, it is suggested that the cannabinoid in compositions described herein, in particular CBD, is effective in treating and preventing inflammation associated with vaginal dryness and vaginal atrophy. Other ingredients such as phytoestrogen provide long-term lubrication, thereby treating vaginal dryness for at least 24 hours. Compositions described herein contain no estrogen and therefore it is suggested that they do not cause the potential side effects associated with vaginal administration of estrogen.

### Example 5

### Testing Efficacy of CBD Cream/Gel

Cream and gel described as in Examples 1-4 is tested using the following method.

An objective of the study is to determine the capacity for improvement after using the product. Women between 30 and 65 years old are tested for 28 days using any of the compositions described herein. The volunteers each apply composition, once daily, using a finger, to the affected area. The amount administered is between 1-5 per daily administration. 30 volunteers are enlisted. Gynecological evaluations are performed at Day 0 (before administration, Day 30, and Day 60 from administration. A dermatological evaluation is performed to verify skin tolerance and product efficacy. The clinical evaluation includes the following parameters: gynecological scoring, pH, vaginal dryness, volume lips (both) and recovery of vaginal tone and elasticity. The products administered were evaluated for the following parameters: possible adverse reactions, itching, flaking and dryness. A quality of life questionnaire as well as subjective questionnaire on the perception of the product is administered, to test the following parameters: discomfort when wearing tight clothing or playing sports, pain, itching, sensation during intimate relationships, urinary incontinence, bladder prolapse.

It is suggested that compositions described herein can improve any of the subjective and objective parameters tested above.

According to an embodiment, described herein is a composition in the form of an emulsion, a moisturizing gel, a cream, a lotion, an oil, a liquid or a lubricating gel, ointment, balm comprising a cannabinoid and a phytoestrogen. Optionally, the composition further comprises a prebiotic agent or a lubricating agent. Optionally, cannabinoid is present from between 0.01% to 5% by weight. Optionally, the cannabinoid is present from between 1% to 2% by weight. Optionally, wherein the phytoestrogen is present in an amount of between 0.003% and 0.15% by weight. Optionally, the prebiotic agent is an α-glucan oligosaccharide. Optionally, the α-glucan oligosaccharide is present in an amount of between 0.5% by weight to 5% by weight. Optionally, wherein the lubricating agent is hyaluronic acid. Optionally, the hyaluronic acid is present in an amount between 0.01% by weight to 1% by weight. Optionally, the hyaluronic acid is present in an amount between 0.2% by weight to 0.5% by weight. Optionally, the cannabinoid is cannabidiol. Optionally, the composition is for use in treatment of vaginal dryness, or vaginal atrophy. Optionally, the composition is for use in a post-menopausal woman. Optionally, the composition is for administration in an amount of 1-5 g daily.

Further described herein is a method for treatment of vaginal dryness comprising administering to a patient in need thereof a composition in the form of an emulsion, a moisturizing gel, a cream, a lotion, an oil, a liquid or a lubricating gel, ointment, balm comprising a cannabinoid and a phytoestrogen. Optionally, patient suffers from vaginal atrophy. Optionally, the patient is a post-menopausal woman. Optionally, the composition is administered in an amount between 1-5 g daily.

Further described herein is a method for providing vaginal lubrication comprising administering to a sexual organ an effective amount of the composition. Optionally, the composition is applied to a condom. Optionally, the composition is applied to a vagina. Optionally, the composition is administered via an applicator. Optionally, the composition is administered before or during sexual intercourse.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A composition in the form of an emulsion, a moisturizing gel, a cream, a lotion, an oil, a liquid or a lubricating gel, ointment, balm comprising a cannabinoid and a phytoestrogen.

2. The composition according to claim 1, further comprising a prebiotic agent or a lubricating agent.

3. The composition according to claim 1 wherein the cannabinoid is present from between 0.01% to 5% by weight.

4. The composition according to claim 3 wherein the cannabinoid is present from between 1% to 2% by weight.

5. The composition according to claim 1 wherein the phytoestrogen is present in an amount of between 0.003% and 0.15% by weight.

6. The composition according to claim 2 wherein the prebiotic agent is an α-glucan oligosaccharide.

7. The composition according to claim 6 wherein the α-glucan oligosaccharide is present in an amount of between 0.5% by weight to 5% by weight.

8. The composition according to claim 2 wherein the lubricating agent is hyaluronic acid.

9. The composition according to claim 8 wherein the hyaluronic acid is present in an amount between 0.01% by weight to 1% by weight.

10. The composition according to claim 9 wherein the hyaluronic acid is present in an amount between 0.2% by weight to 0.5% by weight.

11. The composition according to claim 1 wherein the cannabinoid is cannabidiol.

12. A method for treatment of vaginal dryness in a patient in need thereof, comprising administering to a patient in need thereof a composition according to claim 1.

13. The method according to claim 12 wherein the patient suffers from vaginal atrophy.

14. The method according to claim 13 wherein the patient is a post-menopausal woman.

15. The method according to claim 12 wherein the composition is administered in an amount between 1-5 g daily.

16. A method for providing vaginal lubrication comprising administering to a sexual organ an effective amount of a composition according to claim 1.

17. The method according to claim 16 wherein the composition is applied to a condom.

18. The method according to claim 16 wherein the composition is applied to a vagina.

19. The method according to claim 16 wherein the composition is administered via an applicator.

20. The method according to claim 12 wherein the composition is administered before or during sexual intercourse.

21-24. (canceled)
